(19) United States
(12) Patent Application Publication
(10) Pub. No.: US 2003/0009062 A1
(43) Pub. Date: Jan. 9, 2003

(54) Method for producing trimethylol alkanes

(76) Inventors: Frank Dobert, Koln (DE); Alexander Klausener, Pulheim (DE); Paul Wagner, Dusseldorf (DE)

Correspondence Address: BAYER CORPORATION, PATENT DEPARTMENT, 100 BAYER ROAD, PITTSBURGH, PA, 15205, US

(21) Appl. No.: 10/181,011

(22) PCT Filed: Jan. 3, 2001
(86) PCT No.: PCT/EP01/00016

(30) Foreign Application Priority Data
Jan. 14, 2000 (DE) 100 01 257.4

(51) Int. Cl. C07C 29/141; C07C 53/06

(52) U.S. Cl. 568/853; 562/609

(57) ABSTRACT

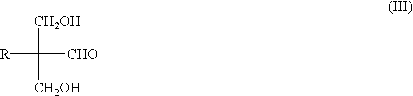

The invention relates to a two-step method for producing trimethylol alkanes of the general formula (I), (HOCH₂)₃—C—R, wherein R represents methylol C₁-C₁₂ alkyl, C₆-C₁₀ aryl or C₁-C₂₂ aralkyl, and simultaneously producing calcium formiate on the basis of an aldehyde of the formula (II), RCH₂CHO, wherein R has the meaning indicated above. In a first step, an aldehyde of the formula (II) and a formaldehyde are reacted in the presence of a base to give a 2,2-dimethylol alkanal of formula (III), wherein R has the meaning indicated above. In a second step, the compound of formula (III) is reacted with formaldehyde in the presence of calcium hydroxide. The invention provides a method for obtaining trimethylol alkanes of the general formula (I) with a high purity and in high yields.

The present invention relates to a process for preparing trimethylolalkanes, in particular trimethylolpropane, in high purity and in high yields with simultaneous production of calcium formate (Ca(OOCH)₂).

Both trimethylolalkanes and calcium formate are industrially useful products. Thus, trimethylolpropane is used in the production of surface coating resins, powder coatings, foams and polyesters. Calcium formate is used commercially in, for example, the following fields: Additive for animal nutrition, use in the building materials industry, preparation of formic acid, auxiliary in the leather industry, auxiliary in the production of high-gloss paper, treatment of scrubbing water in flue gas desulfurization and auxiliary in silage production.

The industrial preparation of trimethylolpropane (TMP) employs n-butyraldehyde and formaldehyde as starting materials. It is generally agreed that 2,2-dimethylolbutanal is formed first in a base-catalyzed reaction via the intermediate 2-methylolbutanal. In the final step in the presence of stoichiometric amounts of a base, for example calcium hydroxide, trimethylolpropane is formed with simultaneous liberation of calcium formate. The process is carried out as a single-stage process, which has the disadvantage that the individual reaction steps, i.e. the formation of 2,2-dimethylolbutanal and its conversion into trimethylolpropane, cannot be optimized separately. This is reflected in the formation of undesirable by-products and in an unsatisfactory yield based on the n-butyraldehyde used.

To avoid this disadvantage, two-stage processes in which 2,2-dimethylolbutanal is firstly prepared from n-butyraldehyde and formaldehyde in a first step and this is then hydrogenated in a second step have been developed.

DE-A 25 07 461 describes, for example, a two-stage process in which 2,2-dimethylolbutanal is obtained from n-butyraldehyde and formaldehyde in the presence of catalytic amounts of a tertiary trialkylamine bearing at least one branched alkyl radical and can then be subjected to hydrogenation. The yields of trimethylolpropane of about 75%, based on n-butyraldehyde used, are unsatisfactory.

According to DE-A 196 53 093 the yield of trimethylolpropane, both based on the n-butyraldehyde used and based on the formaldehyde used, can be significantly increased if, in a first step, the preparation of 2,2-dimethylolbutanal is carried out by condensation of n-butyraldehyde and formaldehyde in the presence of catalytic amounts of a tertiary amine in three stages, with unreacted starting material and by-products formed being recycled and reacted further. The condensation product obtained in this way (2,2-dimethylolbutanal) is hydrogenated to trimethylolpropane in a second step.

EP-A 860 419, too, proposes carrying out the preparation of 2,2-dimethylolbutanal from n-butyraldehyde and formaldehyde, i.e. the first step in the preparation of trimethylolpropane, in a plurality of stages, with the actual reaction being carried out in the first stage and the 2-ethylacrolein formed as by-product being reacted with further formaldehyde in the second stage. The 2,2-dimethylolbutanal prepared in this way can be hydrogenated in a second step to give trimethylolpropane.

The major advantage of the above-described process variants for preparing trimethylolpropane in two steps, namely the preparation of 2,2-dimethylolbutanal and subsequent preparation of trimethylolpropane, is that both steps can be optimized individually and good yields can thus be achieved. However, this is countered by serious disadvantages. Firstly, calcium formate is not obtained. Secondly, the necessary hydrogenation is generally carried out under superatmospheric pressure, which requires the use of expensive pressure-rated reactors. In addition, the 2,2-dimethylolbutanal obtained in the first step has to be largely free of unreacted starting material, in particular formaldehyde, and basic constituents before the hydrogenation step for the desired high yields of trimethylolpropane to be achieved.

It is an object of the present invention to provide a process for preparing trimethylolalkanes in high yields based on the starting materials used, which allows the simultaneous production of calcium formate.

We have now found a process for preparing trimethylolalkanes of the general formula I

(HOCH₂)₃—C—R I

where

R represents methylol, C₁-C₁₂-alkyl, C₆-C₁₀-aryl or C₇-C₂₂-aralkyl,

with simultaneous production of calcium formate starting from an aldehyde of the formula II

RCH₂CHO II,

where

R is as defined above,

which is characterized in that an aldehyde of the formula II and formaldehyde are reacted in the presence of a base to form a 2,2-dimethylolalkanal of the formula III

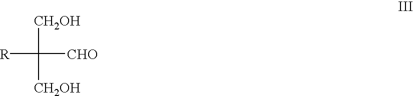

where

R is as defined above,

in a first step and the compound of the formula m is reacted with formaldehyde in the presence of calcium hydroxide in a second step.

In the formulae I, II and m, R represents methylol, C₁-C₁₂-alkyl, for example methyl, ethyl, propyl, butyl, pentyl, hexyl, isopropyl, isobutyl or tert-butyl, C₆-C₁₀-aryl, for example phenyl or naphthyl, or C₇-C₂₂-aralkyl, for example benzyl. R preferably represents methylol or C₁-C₆-alkyl, particularly preferably methylol or C₁-C₃-alkyl. R very particularly preferably represents ethyl.

The process of the invention separates the preparation of the intermediate 2,2-dimethylolalkanal from the subsequent step, namely the preparation of trimethylolalkane, both in a process engineering and a spatial respect. This allows the separate optimization of both process steps. The process of the invention makes it possible to prepare trimethylolalkane in good yield and at the same time obtain calcium formate. Surprisingly, the presence of incompletely reacted 2-methylolalkanal, which is formed as intermediate, in the 2,2-dimethylolalkanal prepared in the first step does not have an adverse effect on yield and selectivity in respect of the formation of trimethylolalkanes. A further surprising aspect is that, unlike the classical single-stage process variant, the second step of the process of the invention, namely the formation of trimethylolalkane from 2,2-dimethylolalkanal in the presence of calcium hydroxide and formaldehyde, forms only very small amounts of by-products. This second reaction step of the process of the invention proceeds surprisingly selectively. Products of neither a mixed Cannizzaro reaction nor a retro-aldol reaction, i.e. decomposition of the 2,2-dimethylolalkanal, are observed. The formation of compounds having relatively high molecular weights, e.g. 2-ethyl-2-{[2-ethyl-2-(hydroxymethyl)butoxy]-methyl}-1,3-propanediol and 2,2-bis(hydroxymethyl)butyl formate, is also observed to only a small extent.

In the first step of the process of the invention, an aldehyde of the formula II is reacted with formaldehyde in the presence of a base. This reaction is known per se to those skilled in the art and is advantageously carried out in a plurality of stages, for example as described in DE-196 53 093 and EP-A 860 419.

In this step, the aldehyde of the formula II is preferably used in the form of an aqueous solution. For example, it is used directly in the form in which it is obtained from its production by customary industrial processes.

Formaldehyde is preferably used in the form of an aqueous solution containing from about 1 to 55% by weight, preferably from 5 to 35% by weight, particularly preferably from 10 to 32% by weight, of formaldehyde.

The molar ratio of aldehyde of the formula II to formaldehyde can be, for example, from 1:2 to 1:10, preferably from 1:2 to 1:5, particularly preferably from 1:2 to 1:3.5.

Suitable bases are, for example, those which are known as basic catalysts for the aldol condensation. Particularly useful bases are alkali metal hydroxides and alkaline earth metal hydroxides, alkali metal hydrogencarbonates and alkaline earth metal hydrogencarbonates, alkali metal carbonates and alkaline earth metal carbonates and tertiary amines. Preference is given to sodium hydroxide, calcium hydroxide, sodium hydrogencarbonate, sodium carbonate and trialkylamines having from 1 to 6 carbon atoms per alkyl group, particularly preferably sodium hydroxide, calcium hydroxide and trialkylamines having from 1 to 4 carbon atoms per alkyl group and very particularly preferably calcium hydroxide and trialkylamines having from 1 to 2 carbon atoms per alkyl group, with trimethylamine and triethylamine deserving special mention. It is possible to use either one base or a mixture of two or more bases.

In the first step of the process of the invention, the bases can be used, for example, in an amount of from 0.001 to 0.5 mol per mole of aldehyde of the formula II. Preference is given to from 0.01 to 0.4 mol of base per mole of aldehyde, particularly preferably from 0.05 to 0.2 molar equivalents.

The concentration of the organic components in the reaction mixture can be, for example, from 5 to 50% by weight, preferably from 10 to 40% by weight.

The reaction can, for example, be carried out at a temperature of from 0 to 130° C., preferably from 10 to 100° C., particularly preferably from 10 to 80° C. If the chosen reaction temperature exceeds the boiling point of the components of the reaction mixture, the first step of the process of the invention can be carried out under superatmospheric pressure.

A particularly high space-time yield and a high yield of 2,2-dimethylolalkanal of the formula III can be achieved by means of a particular reaction temperature profile. The first step of the process of the invention is therefore preferably commenced at a relatively low temperature, for example at from 0 to 60° C., and the temperature is then increased continuously or stepwise to a final temperature which should not exceed 130° C. The desired final temperature can, for example, be reached after a time of from 10 minutes to 3 hours.

In a preferred embodiment of the first step of the process of the invention, a pH of the reaction mixture in the range from 8 to 12 is set. The pH can be adjusted by addition of the abovementioned bases. For this purpose, it can become necessary to add the base in a plurality of successive portions.

The residence time of the reaction mixture in the reactor can be, for example, from 10 minutes to 10 hours.

The process can be carried out batchwise, semibatchwise or continuously. Possible reaction apparatuses are all reaction apparatuses known to those skilled in the art which are suitable for the reaction of liquid reactants. Particular mention may be made of the stirred tank reactor, the cascade of stirred tank reactors, the flow tube and the multichamber reactor or the combination of these apparatuses.

The first step of the process of the invention is preferably continued to a conversion of only 40-80%, preferably 50-70%, defined as the molar ratio of aldehyde of the formula II reacted to aldehyde of the formula II used, and the unreacted aldehyde of the formula II together with any 2-substituted acrylaldehyde formed as by-product is separated from the reaction mixture. The separation can be carried out by means of a phase separation in which the organic phase containing essentially aldehyde of the formula II, 2-methylolalkanal and the 2-substituted acrylaldehyde is separated from the aqueous phase containing predominantly 2,2-dimethylolalkanal of the formula III and formaldehyde. The organic phase which has been separated off is recycled. If desired, all or some of the organic phase can be subjected to distillation prior to recycling, with the distillate formed being recycled. As an alternative to phase separation, the separation can also be carried out by distillation. This distillation is preferably carried out as a rectification, for example batchwise or continuously. The rectification can, for example, be carried out at a pressure of from 0.01 to 50 bar, preferably from 0.1 to 10 bar. The organic phase to be recycled or its distillate can be returned directly to the first reaction stage or can firstly be pretreated in a separate reaction stage, as is known from DE-A 196 53 093 and EP-A 860 419.

The first step of the process of the invention results in 2,2-dimethylolalkanal of the formula III, generally in a yield of >90%, preferably >95%, based on aldehyde of the formula II used. 2,2-Dimethylolalkanal is present in the aqueous phase of the reaction mixture formed. The content of 2,2-dimethylolalkanal of the formula III in the aqueous phase is preferably 5-60% by weight, preferably 15-40% by weight.

The 2,2-dimethylolalkanal of the formula m can be isolated if desired, for example by distillation. However, preference is given to separating off the aqueous phase from the first reaction step and, without isolating the 2,2-dimethylolalkanal of the formula m, passing it to the second step of the process of the invention.

In the second step of the process of the invention, the 2,2-dimethylolalkanal of the formula III obtained from the first step is reacted with calcium hydroxide and formaldehyde to give the corresponding trimethylolalkane of the formula I. In this second step, the 2,2-dimethylolalkanal of the formula III is preferably used in aqueous solution.

The molar ratio of 2,2-dimethylolalkanal of the formula III to formaldehyde can be, for example, from 1:1 to 1:5, preferably from 1:1 to 1:3, particularly preferably from 1:1 to 1:1.5.

The formaldehyde is preferably used in the form of an aqueous solution containing, for example, from 1 to 55% by weight, preferably from 5 to 35% by weight, particularly preferably from 10 to 32% by weight, of formaldehyde.

In a preferred variant of the process of the invention, the aqueous solution of 2,2-dimethylolalkanal of the formula III obtained from the first reaction step contains incompletely reacted formaldehyde and/or formaldehyde which has not yet been completely separated off. If such solutions are used in the second reaction step, correspondingly less formaldehyde has to be added to set the molar ratios indicated above. For example, the first reaction step can be carried out using an excess of formaldehyde, preferably an excess chosen so that no further formaldehyde has to be added in the second reaction step. The problematical removal of formaldehyde from an aqueous solution in the presence of a 2,2-dimethylolalkanal of the formula III is dispensed with, which represents a further advantage of the process since the selectivity of the first reaction step increases with the formaldehyde excess.

The amount of calcium hydroxide added can be, for example, from 0.4 to 1 molar equivalents, preferably from 0.5 to 0.7 molar equivalents, particularly preferably from 0.5 to 0.6 molar equivalents, based on the 2,2-dimethylolalkanal of the formula III.

The second step of the process of the invention can be carried out, for example, at temperatures of from 10 to 130° C., preferably from 10 to 80° C., particularly preferably from 10 to 70° C. If the chosen reaction temperature exceeds the boiling point of the components of the reaction mixture, the second step of the process of the invention can be carried out under superatmospheric pressure.

This step can be carried out continuously, semibatchwise or batchwise in known reaction apparatuses, e.g. stirred tank reactors, cascades of stirred tank reactors or multichamber reactors or a combination of these apparatuses.

The residence time in the reactor can be, for example, from 5 minutes to 10 hours, preferably from 10 minutes to 5 hours.

If 2-methylolalkanal is present as secondary component in the aqueous solution of the 2,2-dimethylolalkanal of the formula III from the first reaction step, this does not have an adverse effect on the second step. Under the conditions of the 2nd reaction step, 2-methylolalkanal is likewise converted into the desired trimethylolalkane. If 2-methylolalkanal is present in the 2,2-dimethylolalkanal solution, the 2-methylolalkanal present has to be added to the 2,2-dimethylolalkanal of the formula III in the above figures for molar ratios of 2,2-dimethylolalkanal to formaldehyde and to calcium hydroxide.

The process of the invention results in an aqueous suspension containing essentially trimethylolalkane of the formula I together with the calcium formate formed and unreacted formaldehyde.

The reaction products trimethylolalkane of the formula I and calcium formate can be isolated in pure form in a manner known per se.

The process of the invention has been found to be particularly advantageous for the preparation of trimethylolpropane from n-butyraldehyde and formaldehyde.

The following examples serve to illustrate the process of the invention, but the process is in no way restricted to the examples.

### EXAMPLES

The preparation of 2,2-dimethylolalkanal is known. For example, n-butyraldehyde and formaldehyde can be reacted in the presence of catalytic amounts of a tertiary amine to give 2,2-dimethylolbutanal, as described in DE-A 196 53 093. The 2,2-dimethylolbutanal obtained in this way can be used in the second step of the process of the invention. However, it is also possible to carry out the second step of the process of the invention using 2,2-dimethylolalkanal solutions which have been prepared by other known methods. The following examples demonstrate that trimethylolpropane is obtained in yields of greater than 93% when aqueous 2,2-dimethylolbutanal solutions are employed in the second step of the process of the invention.

### EXAMPLE 1

9.11 g of calcium hydroxide (0.123 mol) together with 148.6 g of water were placed in a 0.5 l glass reactor and the mixture was heated to 50° C. 163.3 g of an aqueous 2,2-dimethylolbutanal solution containing 19.1% by weight of 2,2-dimethylolbutanal (0.236 mol), 1.7% by weight of trimethylolpropane (0.021 mol) and 10.2% by weight of formaldehyde (0.555 mol) were then added dropwise to this suspension over a period of 15 minutes. The reaction mixture was subsequently allowed to react further for 10 minutes. The product solution contained 10.13% by weight of trimethylolpropane (yield: 94.3% of theory).

### EXAMPLE 2

4.56 g of calcium hydroxide (0.062 mol) together with 61.0 g of water were placed in a 0.5 l glass reactor and the mixture was heated to 40° C. 100 g of an aqueous 2,2-dimethylolbutanal solution containing 16.4% by weight of 2,2-dimethylolbutanal (0.124 mol), 0.9% by weight of trimethylolpropane (0.67 mol) and 12.1% by weight of formaldehyde (0.403 mol) were then added dropwise to this suspension over a period of 15 minutes. The reaction mixture was subsequently allowed to react further for 20 minutes. The product solution contained 9.73% by weight of trimethylolpropane (yield: 96.3% of theory).

### EXAMPLE 3

4.56 g of calcium hydroxide (0.062 mol) together with 60.0 g of water were placed in a 0.5 l glass reactor and the mixture was heated to 25° C. 100 g of an aqueous 2,2-dimethylolbutanal solution containing 16.1% by weight of 2,2-dimethylolbutanal (0.122 mol), 2.91% by weight of trimethylolpropane (0.022 mol) and 12.7% by weight of formaldehyde (0.424 mol) were then added dropwise to this suspension over a period of 15 minutes. The reaction mixture was subsequently allowed to react further for 60 minutes. The product solution contained 11.24% by weight of trimethylolpropane (yield: 95.7% of theory).

### EXAMPLE 4

4.56 g of calcium hydroxide (0.062 mol) together with 60.0 g of water were placed in a 0.5 l glass reactor and the mixture was heated to 30° C. 100 g of an aqueous 2,2-dimethylolbutanal solution containing 16.1% by weight of 2,2-dimethylolbutanal (0.122 mol), 2.91% by weight of trimethylolpropane (0.022 mol) and 12.7% by weight of formaldehyde (0.424 mol) were then added dropwise to this suspension over a period of 15 minutes. The reaction mixture was subsequently allowed to react further for 20 minutes. The product solution contained 10.99% by weight of trimethylolpropane (yield: 93.6% of theory).

1. A process for preparing trimethylolalkanes of the general formula I
(HOCH₂)₃—C—R I
where
R represents methylol, C₁-C₁₂-alkyl, C₆-C₁₀-aryl or C₇-C₂₂-aralkyl,
with simultaneous production of calcium formate starting from an aldehyde of the formula II
RCH₂CHO II,
where
R is as defined above,
characterized in that an aldehyde of the formula In and formaldehyde are reacted in the presence of a base to form a 2,2-dimethylolalkanal of the formula III 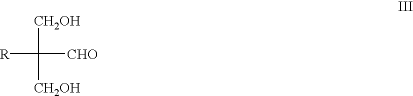
where
R is as defined above,
in a first step and the compound of the formula III is reacted with formaldehyde in the presence of calcium hydroxide in a second step.

2. The process as claimed in claim 1, characterized in that R represents methylol or C₁-C₃-alkyl.

3. The process as claimed in at least one of claims 1 and 2, characterized in that trimethylolpropane is prepared starting from n-butyraldehyde.

4. The process as claimed in at least one of claims 1 to 3, characterized in that the first reaction step is carried out in stages with recycling of by-products formed and unreacted starting materials.

5. The process as claimed in at least one of claims 1 to 4, characterized in that a 2- to 10-fold molar amount of formaldehyde, based on the aldehyde of the formula II, is used in the first step.

6. The process as claimed in at least one of claims 1 to 5, characterized in that the base used in the first reaction step is trimethylamine, triethylamine, sodium hydroxide and/or calcium hydroxide.

7. The process as claimed in at least one of claims 1 to 6, characterized in that the base used in the first reaction step is present in an amount of from 0.001 to 0.5 mol per mole of aldehyde of the formula II.

8. The process as claimed in at least one of claims 1 to 7, characterized in that the aldehyde of the formula m used in the second reaction step is used in the form of an aqueous solution having an aldehyde content of 5-60% by weight.

9. The process as claimed in at least one of claims 1 to 8, characterized in that the second reaction step is carried out using from 0.4 to 1 molar equivalents of calcium hydroxide, based on the aldehyde of the formula III.

10. The process as claimed in at least one of claims 1 to 9, characterized in that the molar ratio of 2,2-dimethylolalkanal of the formula III to formaldehyde in the second reaction step is from 1:1 to 1:5.
